(19) United States
(12) Patent Application Publication
(10) Pub. No.: US 2020/0261483 A1
(43) Pub. Date: Aug. 20, 2020

(54) METHODS AND COMPOSITIONS FOR TREATING CYSTIC FIBROSIS

(71) Applicant: Elysium Health, Inc., New York, NY (US)

(72) Inventors: Eric Marcotulli, New York, NY (US); Daniel Alminana, New York, NY (US); Ryan Dellinger, Azusa, CA (US); Mark Morris, New York, NY (US)

(21) Appl. No.: 16/760,317

(22) PCT Filed: Oct. 29, 2018
(86) PCT No.: PCT/US18/57924

§ 371 Date: Apr. 29, 2020

Related U.S. Application Data
(60) Provisional application No. 62/578,559, filed on Oct. 30, 2017.

(51) Int. Cl. A61K 31/706; A61K 31/09

(52) U.S. Cl. CPC A61K 31/706; A61K 45/06; A61K 31/09

(57) ABSTRACT

Provided herein are methods and compositions related to treating and/or preventing cystic fibrosis in a subject by administering to the subject (e.g., orally administering to the subject) a composition comprising nicotinamide riboside and/or pterostilbene.

### CROSS-REFERENCE TO RELATED APPLICATIONS

This application claims the benefit of U.S. Provisional Application No. 62/578,559, filed on Oct. 30, 2017; hereby incorporated by reference in its entirety.

### BACKGROUND

Cystic fibrosis is an inherited disease characterized by the buildup of thick, sticky mucus that can damage many of the body's organs. The disorder's most common signs and symptoms include progressive damage to the respiratory system and chronic digestive system problems. The features of the disorder and their severity varies among affected individuals.

Mucus is a slippery substance that lubricates and protects the linings of the airways, digestive system, reproductive system, and other organs and tissues. In people with cystic fibrosis, the body produces mucus that is abnormally thick and sticky. This abnormal mucus can clog the airways, leading to severe problems with breathing and bacterial infections in the lungs. These infections cause chronic coughing, wheezing, and inflammation. Over time, mucus buildup and infections result in permanent lung damage, including the formation of scar tissue (fibrosis) and cysts in the lungs. Most patients with cystic fibrosis also have digestive problems, such as intestinal blockage from mucus buildup. Other digestive problems result from a buildup of thick, sticky mucus in the pancreas. In cystic fibrosis patients, mucus blocks the ducts of the pancreas, reducing the production of insulin and preventing digestive enzymes from reaching the intestines to aid digestion. Problems with digestion can lead to diarrhea, malnutrition, poor growth, and weight loss. In adolescence or adulthood, a shortage of insulin can cause a form of diabetes known as cystic fibrosis-related diabetes mellitus (CFRDM). There is no cure for cystic fibrosis. Consequently, there is a need for novel and improved therapies for cystic fibrosis and cystic fibrosis-related disorders.

### SUMMARY

In some aspects, the methods and compositions disclosed herein relate to treating cystic fibrosis and cystic fibrosis transmembrane conductance regulator (CFTR)-related diseases or disorders. Provided herein are methods and compositions related to treating cystic fibrosis or a CFTR-related disease or disorder (e.g., pancreatitis and/or bronchiectasis) and/or for treating and/or preventing a symptom of cystic fibrosis or a symptom of CFTR-related disease or disorder in a subject in need thereof, by administering to the subject (e.g., orally administering to the subject) a composition comprising a compound of Formula I or Formula II (e.g., nicotinamide riboside), and/or a compound of Formula III (e.g., pterostilbene).

The symptom may be a respiratory symptom (e.g., sticky mucus buildup in lungs, atelectasis, lung infections, chronic cough, wheezing, collapsed lung, nasal polyps, clubbing of finger or toes, and/or sinusitis), a digestive symptom (e.g., bowel obstruction, fatty stools, severe constipation, rectal prolapse, pancreatitis, and/or biliary cirrhosis), or a symptom affecting the reproductive tract (e.g., blockage of the vas deferens).

Also provided herein are methods of increasing chloride ion channel transport in epithelial cells (e.g., epithelial cells or tissue in the lining of the lungs, digestive tract, or reproductive tract) of a subject with cystic fibrosis or a CFTR-related disease or disorder, comprising by administering to the subject (e.g., orally administering to the subject) a composition comprising a compound of Formula I or Formula II (e.g., nicotinamide riboside), and/or a compound of Formula III (e.g., pterostilbene).

In some aspects, provided herein are methods of reducing sticky mucus in the lungs, digestive tract, and/or reproductive tract in a subject with cystic fibrosis or a CFTR-related disease or disorder by administering to the subject (e.g., orally administering to the subject) a composition comprising a compound of Formula I or Formula II (e.g., nicotinamide riboside), and/or a compound of Formula III (e.g., pterostilbene). In some embodiments, the subject carries a CFTR gating mutant allele or a CFTR conductance mutant allele.

In some embodiments, the methods comprise conjointly administering a second therapeutic for the treatment of cystic fibrosis, such as a CFTR potentiator, a mucoactive agent, an antibiotic, an inhaled beta agonist, and/or an anti-inflammatory agent.

In certain embodiments of the compositions and methods provided herein, the composition comprises a compound of Formula I or Formula II (e.g., nicotinamide riboside) (e.g., at least 100 mg, at least 125 mg, at least 150 mg, at least 175 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 525 mg, at least 550 mg, at least 575 mg or at least 600 mg of a compound of Formula I or Formula II (e.g., nicotinamide riboside)). In some embodiments, the composition comprises a compound of Formula III (e.g., pterostilbene) (e.g., at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg or at least 150 mg of a compound of Formula III (e.g., pterostilbene)). In certain embodiments, the composition comprises both a compound of Formula I or Formula II (e.g., nicotinamide riboside) (e.g., at least 100 mg, at least 125 mg, at least 150 mg, at least 175 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 525 mg, at least 550 mg, at least 575 mg or at least 600 mg of a compound of Formula I or Formula II (e.g., nicotinamide riboside)) and a compound of Formula III (e.g., pterostilbene) (e.g., at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg or at least 150 mg of a compound of Formula III (e.g., pterostilbene)).

In certain embodiments, the method comprises administering a plurality of doses of the composition. In some embodiments, at least 7 doses of the composition are administered. In some embodiments, at least 30 doses of the composition are administered. In some embodiments, at least 60 or more doses of the composition are administered. In some embodiments, each dose comprises at least 100 mg, at least 125 mg, at least 150 mg, at least 175 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 525 mg, at least 550 mg, at least 575 mg or at least 600 mg of a compound of Formula I or Formula II (e.g., nicotinamide riboside). In some embodiments, each dose comprises at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg or at least 150 mg of a compound of Formula III (e.g., pterostilbene). In certain embodiments, each dose comprises at least 100 mg, at least 125 mg, at least 150 mg, at least 175 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 525 mg, at least 550 mg, at least 575 mg or at least 600 mg of a compound of Formula I or Formula II (e.g., nicotinamide riboside) at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg or at least 150 mg of a compound of formula III (e.g., pterostilbene).

In certain embodiments, a dose of the composition is administered at regular intervals over a period of time. In some embodiments, a dose of the composition is administered at least once a week. In some embodiments, a dose of the composition is administered at least twice a week. In certain embodiments, a dose of the composition is administered at least three times a week. In some embodiments, a dose of the composition is administered at least once a day. In some embodiments, a dose of the composition is administered at least twice a day. In some embodiments, doses of the composition are administered for at least 1 week, for at least 2 weeks, for at least 3 weeks, for at least 4 weeks, for at least 1 month, for at least 2 months, for at least 3 months, for at least 4 months, for at least 5 months, for at least 6 months or for at least 1 year.

In certain embodiments, the composition is formulated for oral delivery. In some embodiments, the composition is formulated as a pill, a tablet, or a capsule. In some embodiments, the composition is administered orally. In certain embodiments, the composition is self-administered.

### DETAILED DESCRIPTION

### General

Provided herein are methods and compositions related to treating cystic fibrosis and/or for treating, preventing, and/or improving a symptom cystic fibrosis in a subject in need thereof by administering to the subject (e.g., orally administering to the subject) a composition comprising a compound of Formula I or Formula II (e.g., nicotinamide riboside), and/or a compound of Formula III (e.g., pterostilbene). Also provided herein are methods and compositions for treating a CFTR-related disease or disorder (e.g., pancreatitis and/or bronchiectasis) and/or for treating, preventing, and/or improving a symptom of a CTFR-related disease or disorder in a subject in need thereof, by administering to the subject (e.g., orally administering to the subject) a composition comprising a compound of Formula I or Formula II (e.g., nicotinamide riboside), and/or a compound of Formula III (e.g., pterostilbene).

Also provided herein are methods of increasing chloride ion channel transport in epithelial cells (e.g., lung, digestive, or reproductive epithelial cells and tissue) of a subject with cystic fibrosis by administering to the subject (e.g., orally administering to the subject) a composition comprising a compound of Formula I or Formula II (e.g., nicotinamide riboside), and/or a compound of Formula III (e.g., pterostilbene).

In some aspects, provided herein are methods of reducing sticky mucus in the lungs, reproductive tract, and/or digestive tract of a subject with cystic fibrosis or a CFTR-related disease or disorder by administering to the subject (e.g., orally administering to the subject) a composition comprising a compound of Formula I or Formula II (e.g., nicotinamide riboside), and/or a compound of Formula III (e.g., pterostilbene).

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering. Administration of a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

The phrase “pharmaceutically-acceptable carrier” as used herein means a pharmaceutically-acceptable material, composition or vehicle, such as a liquid or solid filler, diluent, excipient, or solvent encapsulating material.

As used herein, the term “subject” means a human or non-human animal selected for treatment or therapy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

As used herein, a therapeutic that “prevents” a disorder or condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

### Compositions

Provided herein are pharmaceutical compositions comprising a compound of Formula I or Formula II (e.g., nicotinamide riboside) and/or a compound of Formula III (e.g., pterostilbene).

Nicotinamide riboside is a pyridine-nucleoside form of niacin (i.e., vitamin B₃) that serves as a precursor to nicotinamide adenine dinucleotide (NAD⁺). As used herein, “nicotinamide riboside” also includes nicotinamide riboside salts, such as nicotinamide riboside chloride. The chemical structure of nicotinamide riboside is provided below:

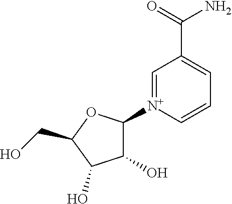 [SMILES: NC(=O)c1ccc[n+]([C@@H]2O[C@H](CO)[C@@H](O)[C@H]2O)c1]

In some embodiments, provided herein are pharmaceutical compositions comprising a compound represented by Formula (I) or a pharmaceutically acceptable salt thereof:

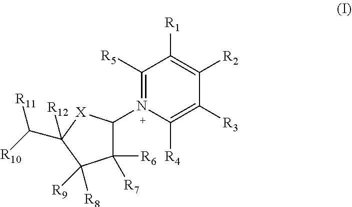

wherein, independently for each occurrence:

R¹, R², and R³ are selected from hydrogen, halogen, —CN, —NO₂, —OR₁₄, —N(R₁₄)ₘ, —R₁₃, substituted or unsubstituted (C₁-C₆)alkyl, cycloalkyl, heterocycloalkyl, aryl, heteroaryl, aralkyl, and heteroaralkyl;

R₄ and R₅ are selected from hydrogen, halogen, —CN, —NO₂, —OR₁₄, —N(R₁₄)ₘ, substituted or unsubstituted (C₁-C₆)alkyl, cycloalkyl, heterocycloalkyl, aryl, heteroaryl, aralkyl, and heteroaralkyl;

R₆, R₈, R₁₁ and R₁₂ are selected from hydrogen, (C₁-C₆)alkyl, —((C₁-C₆)alkylene)N(R₁₄)ₘ, —C(O)((C₁-C₆)alkylene)N(R₁₄)ₘ, cycloalkyl, heterocycloalkyl, aryl, heteroaryl, aralkyl, heteroaralkyl, —OR₁₄, and —N(R₁₄)ₘ;

R₇, R₉, and R₁₀ are selected from —((C₁-C₆)alkylene)N(R₁₄)ₘ, —OR₄, and —N(R₄)ₘ;

R₁₃ is selected from —OR₁₄, —N(R₁₄)ₘ, —C(O)(R₁₄), —C(O)(OR₁₄), —C(O)N(R₁₄)ₘ, —S(O)₂(OR₁₄), —S(O)OR₄, and —S(O)₂N(R₁₄)ₘ;

R₁₄ is selected from hydrogen, (C₁-C₆)alkyl, cycloalkyl, heterocycloalkyl, aryl, heteroaryl, aralkyl, and heteroaralkyl; and

X is O, S, or N(R₁₄);

m is 2 or 3;

provided that at least one of R₁, R₂, and R₃ is R₁₃.

In some embodiments, R₁ is R₁₃. In some embodiments, R₂ is R₁₃. In some embodiments, R₃ is R₁₃.

In some embodiments, R₁₃ is selected from —OR₁₄, —N(R₁₄)ₘ, —C(O)(R₁₄), —C(O)(OR₁₄), and —C(O)N(R₁₄)ₘ. In some embodiments, R₁₃ is selected from —C(O)(R₁₄), —C(O)(OR₁₄), and —C(O)N(R₁₄)ₘ. In some embodiments, R₁₃ is —C(O)N(R₁₄)ₘ.

In some embodiments, R₇, R₉, and R₁₀ are each independently —OR₁₄ or —N(R₁₄)ₘ. In some embodiments, R₇, R₉, and R₁₀ are —OR₄.

In some embodiments, the compound of formula (I) is represented by Formula (II) or a pharmaceutically acceptable salt thereof:

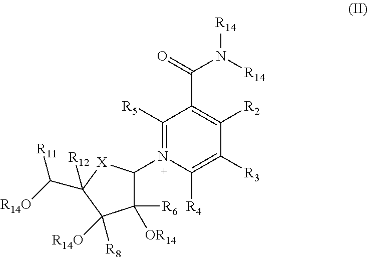

wherein, independently for each occurrence:

R₂ and R₃ are selected from hydrogen, halogen, —CN, —NO₂, —OR₁₄, —N(R₁₄)ₘ, —R₁₃, substituted or unsubstituted (C₁-C₆)alkyl, cycloalkyl, heterocycloalkyl, aryl, heteroaryl, aralkyl, and heteroaralkyl;

R₄ and R₅ are selected from hydrogen, halogen, —CN, —NO₂, —OR₁₄, —N(R₁₄)ₘ, substituted or unsubstituted (C₁-C₆)alkyl, cycloalkyl, heterocycloalkyl, aryl, heteroaryl, aralkyl, and heteroaralkyl;

R₆, R₈, R₁₁, and R₁₂ are selected from hydrogen, —OR₁₄, —N(R₄)ₘ, substituted or unsubstituted (C₁-C₆)alkyl, —((C₁-C₆)alkylene)N(R₁₄)ₘ, —C(O)((C₁-C₆)alkylene)N(R₁₄)ₘ, cycloalkyl, heterocycloalkyl, aryl, heteroaryl, aralkyl, and heteroaralkyl;

R₁₃ is selected from —OR₁₄, —N(R₁₄)ₘ, —C(O)(R₄), —C(O)(OR₄), —C(O)N(R₁₄)ₘ, —S(O)₂(OR₁₄), —S(O)OR₁₄, and —S(O)₂N(R₁₄)ₘ;

R₁₄ is selected from hydrogen, (C₁-C₆)alkyl, cycloalkyl, heterocycloalkyl, aryl, heteroaryl, aralkyl, and heteroaralkyl; and

m is 2 or 3.

In some embodiments of the compounds of formula (I) or (II), R₁, R₂, and R₃ are each independently, if present, selected from hydrogen, halogen, —CN, —NO₂, —OR₁₄, —N(R₁₄)ₘ, —R₁₃, and substituted or unsubstituted (C₁-C₆)alkyl. In some embodiments, R₁, R₂, and R₃ are each independently, if present, selected from hydrogen, —OR₁₄, —N(R₄)ₘ, and unsubstituted (C₁-C₆)alkyl. In some embodiments, R₁, R₂, and R₃ are each independently, if present, selected from substituted or unsubstituted (C₁-C₆)alkyl, cycloalkyl, heterocycloalkyl, aryl, heteroaryl, aralkyl, and heteroaralkyl. In some embodiments, R₁, R₂, and R₃ are each independently, if present, hydrogen.

In some embodiments of the compounds of formula (I) or (II), R₄ and R₅ are each independently selected from hydrogen, halogen, —CN, —NO₂, —OR₁₄, —N(R₁₄)ₘ, and substituted or unsubstituted (C₁-C₆)alkyl. In some embodiments, R₄ and R₅ are each independently selected from hydrogen, —OR₄, —N(R₁₄)ₘ, and unsubstituted (C₁-C₆)alkyl. In some embodiments, R₄ and R₅ are each independently selected from substituted or unsubstituted (C₁-C₆)alkyl, cycloalkyl, heterocycloalkyl, aryl, heteroaryl, aralkyl, and heteroaralkyl. In some embodiments, R₄ and R₅ are each hydrogen.

In some embodiments of the compounds of formula (I) or (II), R₆, R₈, R₁₁, and R₁₂ are selected from hydrogen, —OR₁₄, —N(R₁₄)ₘ, unsubstituted (C₁-C₆)alkyl, —((C₁-C₆)alkylene)N(R₁₄)ₘ, —C(O)((C₁-C₆)alkylene)N(R₁₄)ₘ, cycloalkyl, heterocycloalkyl, aryl, heteroaryl, aralkyl, and heteroaralkyl. In some embodiments, R₆, R₈, R₁₁, and R₁₂ are each independently selected from hydrogen, —OR₁₄, —N(R₁₄)ₘ, unsubstituted (C₁-C₆)alkyl, —((C₁-C₆)alkylene)N(R₁₄)ₘ, and —C(O)((C₁-C₆)alkylene)N(R₁₄)ₘ. In some embodiments, R₆, R₈, R₁₁, and R₁₂ are each independently selected from hydrogen, —OR₁₄, and —N(R₁₄)ₘ. In some embodiments, R₆, R₈, R₁₁, and R₁₂ are each independently selected from unsubstituted (C₁-C₆)alkyl, cycloalkyl, heterocycloalkyl, aryl, heteroaryl, aralkyl, and heteroaralkyl. In some embodiments, R₆, R₈, R₁₁, and R₁₂ are each hydrogen.

In some embodiments, R₇, R₉, and R₁₀ are each independently —OR₄ or —N(R₁₄)ₘ. In some embodiments, R₇, R₉, and R₁₀ are each —OR₁₄. In some embodiments, R₇, R₉, and R₁₀ are each —OH.

In some embodiments of the compounds of formula (I) or (II), R₁₄ is hydrogen or (C₁-C₆)alkyl.

In some embodiments of the compounds of formula (I) or (II), X is O or N(R₁₄). In some embodiments, X is O.

In some embodiments of the compounds of formula (I) or (II), the compound is

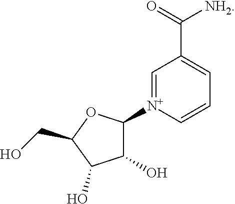 [SMILES: NC(=O)c1ccc[n+]([C@@H]2O[C@H](CO)[C@@H](O)[C@H]2O)c1]

Pterostilbene is a stilbenoid and an analog of polyphenol reservatrol that has better bioavailability due to the presence of two methoxy groups that allow it to have increased lipophilic and oral absorption as well as a longer half-life due to reduced oxidation. The chemical structure of pterostilbene is provided below:

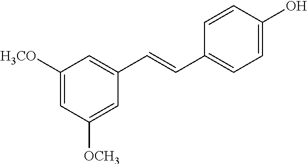 [SMILES: COc1cc(/C=C/c2ccc(O)cc2)cc(OC)c1]

In some embodiments, provided herein are pharmaceutical compositions comprising a compound represented by Formula (III) or a pharmaceutically acceptable salt thereof:

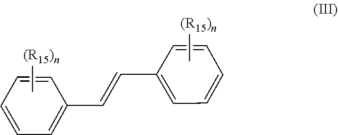

wherein, independently for each occurrence:

R₁₅ is selected from halogen, —CN, —NO₂, —OR₁₆, —N(R₁₆)ₚ, —S(O)₂(OR₁₆), —S(O)OR₁₆, substituted or unsubstituted (C₁-C₆)alkyl, cycloalkyl, heterocycloalkyl, aryl, heteroaryl, aralkyl, and heteroaralkyl;

R₁₆ is selected from hydrogen, (C₁-C₆)alkyl, cycloalkyl, heterocycloalkyl, aryl, heteroaryl, aralkyl, and heteroaralkyl;

n is an integer from 0 to 5; and

p is 2 or 3;

provided that at least one n is 1; and at least one R₁₅ is —OR₁₆;

provided that the compound of formula (III) is not

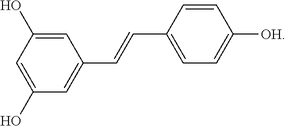 [SMILES: Oc1ccc(/C=C/c2cc(O)cc(O)c2)cc1]

In some embodiments of the compounds of formula (III), R₁₅ is selected from, halogen, —CN, —NO₂, —OR₁₆, —N(R₁₆)ₚ, and substituted or unsubstituted (C₁-C₆)alkyl. In some embodiments, R₁₅ is selected from —OR₁₆, —N(R₁₆)ₚ, and unsubstituted (C₁-C₆)alkyl. In some embodiments, R₁₅ is selected from substituted or unsubstituted (C₁-C₆)alkyl, cycloalkyl, heterocycloalkyl, aryl, heteroaryl, aralkyl, and heteroaralkyl. In some embodiments, R₁₅ is —OR₁₆. In some embodiments, R₁₅ is —OR₁₆; and R₁₆ is hydrogen or (C₁-C₆)alkyl. In some embodiments, R₁₅ is —OR₁₆; and R₁₆ is (C₁-C₆)alkyl, cycloalkyl, heterocycloalkyl, aryl, heteroaryl, aralkyl, and heteroaralkyl. In some embodiments, R₁₅ is —OR₁₆; and R₁₆ is (C₁-C₆)alkyl. In some embodiments, R₁₅ is —OR₁₆; and R₁₆ is (C₁-C₆)alkyl, cycloalkyl, or heterocycloalkyl.

In some embodiments, n is 1, 2, or 3. In some embodiments, n is 1 or 2.

In some embodiments, p is 2. In some embodiments, p is 3.

In one aspect, the provided herein are pharmaceutically acceptable compositions which comprise a therapeutically-effective amount of one or more of the compounds described herein (e.g., nicotinamide riboside and/or pterostilbene), formulated together with one or more pharmaceutically acceptable carriers (additives) and/or diluents. In another aspect, the agents described herein can be administered as such, or administered in mixtures with pharmaceutically acceptable carriers and can also be administered in conjunction with other agents. Conjunctive therapy thus includes sequential, simultaneous and separate, or co-administration of one or more compounds of the invention, wherein the therapeutic effects of the first administered has not entirely disappeared when the subsequent compound is administered.

As described in detail below, the pharmaceutical compositions described herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; or (3) sublingually.

In some embodiments, the composition comprises additional agents. For example, the composition may comprise a nutritional agent, such as an antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The formulations of the compounds described herein may be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the agent which produces a therapeutic effect.

In certain embodiments, a formulation described herein comprises an excipient, including, but not limited to, cyclodextrins, liposomes, micelle forming agents, e.g., bile acids, and polymeric carriers, e.g., polyesters and polyanhydrides; and an agent of the invention. In some embodiments, an aforementioned formulation renders orally bioavailable an agent of the invention. Methods of preparing these formulations or compositions may include the step of bringing into association a compound of the invention with the carrier and, optionally, one or more accessory ingredients.

Liquid dosage forms for oral administration of the formulations provided herein include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations provided herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention as an active ingredient. A compound of the invention may also be administered as a bolus, electuary, or paste.

In solid dosage forms of the invention for oral administration (e.g., capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions described herein, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. Compositions described herein may also be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Pharmaceutical compositions provided herein suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

### Therapeutic Methods

Provided herein are methods and compositions related to treating cystic fibrosis in a subject by administering to the subject (e.g., orally administering to the subject) a composition comprising a compound of Formula I or Formula II (e.g., nicotinamide riboside), and/or a compound of Formula III (e.g., pterostilbene).

Defective ion transport in the lungs, pancreas, gastrointestinal (GI) system, sinuses, skin, and reproductive system leads to the symptoms of cystic fibrosis. The resulting imbalance of fluid and electrolytes causes thick, sticky mucus (e.g., in lungs and/or sinuses) or viscous secretions (e.g., in pancreas, GI tract and/or reproductive tract) to accumulate, which interferes with the proper function of these organs. The gene responsible for cystic fibrosis is CFTR, a cyclic adenosine monophosphate (cAMP)-regulated anion channel expressed at high levels in various epithelia and at much lower levels in many other cell types. As in most other epithelial organs, CFTR in the intestine mediates secretion of chloride, bicarbonate, and fluid. Mutations in the CFTR gene may result in partial or total loss of function, resulting in secreted fluid with abnormally low volume and aberrant electrolyte composition. The altered milieu on the epithelial surface is believed to be the major cause of pathogenesis in affected organs.

Provided herein are methods and compositions related to treating cystic fibrosis and/or for treating, preventing, and/or improving a symptom of cystic fibrosis in a subject in need thereof, by administering to the subject (e.g., orally administering to the subject) a composition comprising a compound of Formula I or Formula II (e.g., nicotinamide riboside), and/or a compound of Formula III (e.g., pterostilbene). Symptoms of cystic fibrosis may include respiratory symptoms, reproductive symptoms, or digestive symptoms.

Symptoms of cystic fibrosis include, but are not limited to, increased mucus in lungs, inefficient clearing of mucus in the lungs, obstruction of airways, atelectasis, infection in the lungs and/or GI tract, bronchitis, inflammation in lungs and/or GI tract, persistent or chronic cough, wheezing, nasal polyps, clubbing of finger or toes, sinusitis, breathlessness, exercise intolerance, greasy/fatty stools, rectal prolapse, poor weight gain and growth, severe constipation, gastrointestinal reflux disease (GERD), pancreatitis, biliary cirrhosis, and/or obstruction of the vas deferens. In some embodiments, the subject has cystic fibrosis-related diabetes. Cystic fibrosis-related diabetes (CFRD) is the most common comorbidity in people with cystic fibrosis (CF), occurring in ˜20% of adolescents and 40-50% of adults. While it shares features of type 1 and type 2 diabetes, CFRD is a distinct clinical entity. It is primarily caused by insulin insufficiency, although fluctuating levels of insulin resistance related to acute and chronic illness also play a role. In some embodiments, the subject has cystic fibrosis-related diabetes. In some embodiments, the methods disclosed herein comprise treating or preventing cystic fibrosis-related diabetes by administering to the subject a compound of Formula I or Formula II (e.g., nicotinamide riboside), and/or a compound of Formula III (e.g., pterostilbene).

The subject may be male or female. In some embodiments, the subject is an adult (i.e., 18 years of age or older). The subject may be pediatric (i.e., less than 18 years of age). In some embodiments, the subject is a mammal, preferably, a human.

In some aspects, provided herein are methods of increasing ion channel transport in epithelial tissue in the lungs, pancreas, gastrointestinal (GI) system, sinuses, and/or reproductive system a subject with cystic fibrosis or a CFTR-related disease or disorder by administering to the subject (e.g., orally administering to the subject) a composition comprising a compound of Formula I or Formula II (e.g., nicotinamide riboside), and/or a compound of Formula III (e.g., pterostilbene).

In some aspects, provided herein are methods of decreasing cystic fibrosis-related inflammation in the lungs, GI tract, pancreas, and/or reproductive tract in a subject in need thereof by administering to the subject (e.g., orally administering to the subject) a composition comprising a compound of Formula I or Formula II (e.g., nicotinamide riboside), and/or a compound of Formula III (e.g., pterostilbene). Also provided herein are methods of decreasing inflammation in the lungs, GI tract, pancreas, and/or reproductive tract in a subject with a CFTR-related disease or disorder by administering to the subject (e.g., orally administering to the subject) a composition comprising a compound of Formula I or Formula II (e.g., nicotinamide riboside), and/or a compound of Formula III (e.g., pterostilbene).

Different mutations in the CFTR gene can cause disruptions at various stages of CFTR protein synthesis or in several aspects of CFTR protein function. They can result in less CFTR protein at the cell surface, absence of CFTR protein, or dysfunctional CFTR protein at the cell surface. In some embodiments, the subject has a mutation in the CFTR gene that affects the quantity (i.e., the number of CFTR channels at the cell surface) and/or function (i.e., the functional ability of each channel to open and transport ions) of the CFTR protein.

The CFTR mutation class system groups CFTR mutations by the primary molecular defect in the CFTR protein. Although each mutation is categorized by a single defect, an individual mutation can result in multiple defects, spanning multiple classes. Mutations in the CFTR gene may be classified into six classes: I-VI. The subject may have a class I (protein production mutation), class II (protein processing mutation), class III (a gating mutation), class IV (conduction mutations), class V (insufficient protein mutations), and/or a class VI mutation (reduced stability mutation). Class I mutations, or protein production mutations, which include nonsense and splice mutations, interfere with the production of the CFTR protein. Examples of class I mutations include premature termination codons and/or nonsense codons. Examples include G542X, W1282X, R553X, 621+G>T. Class II mutations occur when a mutation causes an amino acid to be deleted or an incorrect amino acid to be added, and the CFTR protein cannot form its tertiary structure and function properly. Class 2 mutations include F508del, N1303K, and A455E. Class III mutations, or gating mutations, lock the chloride ion channel in a closed position, decreasing ion exchange across the channel. Examples of class 3 mutations, include, but are not limited to, G551D, G1244E, G1349D, G178R, G551S, S1251N, S1255P, S549N, and S549R. Class IV mutations, or conduction mutations, causes a slowing or inefficient transport of ions across the channel. Examples of such proteins include R117H. Class V mutations, or insufficient protein mutations, result in a reduced amount of normal CFTR protein at the cell surface. Examples of class 5 mutations include, but are not limited to, 3849+10kbC->T, 3272-26A->G, E831X, 2789+5G->A, and 711+3A->G. Class VI mutations include mutations that cause enhanced turnover of CFTR due to C-terminus abnormalities, such mutations include, but are not limited to, 120de1123, rPhe580del and Q1412X. A subject may have any CFTR mutation disclosed herein. The subject may have a class I mutation. The subject may have a class II mutation. The subject may have a class III mutation. The subject may have a class IV mutation. The subject may have a class V mutation. The subject may have a class VI mutation. Finally, the subject may have a mutation resulting in residual function of CTFR, such as E56K, R347H, A1067T, P67L, R352Q, G1069R, R74W,A455E, R1070Q, D110E, D579G, R1070W, D110H, S945L, F1074L, R117C, S977F, D1152H, E193K, F1052V, D1270N, L206W, or K1060T. A subject may have any CFTR mutation disclosed herein or any CFTR mutation that causes a pathological phenotype. A list of CFTR mutations can be found in US CF Foundation, Johns Hopkins University, The Hospital for Sick Children. The Clinical and Functional Translation of CFTR (CFTR2) (http://www.cftr2.org.), hereby incorporated by reference in its entirety.

In some embodiments, a composition comprising a compound of Formula I or Formula II (e.g., nicotinamide riboside), and/or a compound of Formula III (e.g., pterostilbene) may be administered conjointly with a second therapy or therapeutic, such as a CTFR modulator therapeutic. Examples of such therapeutics include ivacaftor or the combination drug lumacaftor/ivacaftor. Ivacaftor is a drug used to treat cystic fibrosis in people with certain mutations in the cystic fibrosis transmembrane conductance regulator (CFTR) gene. The methods provided herein may be administered in conjointly with additional therapies for cystic fibrosis, including, but not limited to, a mucoactive agent, an antibiotic, an inhaled beta-agonist, or an anti-inflammatory agent.

In some embodiments, provided herein are methods of treating a CFTR-related disorder and/or improving a symptom of a CFTR-related disorder. A CFTR-related disease or disorder is a clinical diagnosis that is associate with CFTR dysfunction but do not fulfill the diagnostic criteria for cystic fibrosis. Examples of a CFTR-related diseases or disorders include congenital bilateral absence of the vas deferens (CBAVD), acute, recurring, or chronic pancreatitis, and acute, reoccurring or chronic bronchiectasis. A symptom of a CFTR-related disease or disorder may be any symptom of cystic fibrosis disclosed herein.

Actual dosage levels and administration regimen of the compositions disclosed herein may be varied so as to obtain an amount of a compound of Formula I or Formula II (e.g., nicotinamide riboside) and/or a compound of Formula III (e.g., pterostilbene) that is effective to achieve the desired therapeutic response for a particular patient, composition, and mode of administration, without being toxic to the patient. In some embodiments, the subject continuously self-administers the compounds disclosed herein. In other embodiments, the subject may take a compound disclosed herein as needed.

In some embodiments, administration of the composition comprises administration of the composition in one or more dose(s). In some embodiments, administration of the composition comprises administration of the composition in one or more, five or more, ten or more, twenty or more, thirty or more, forty or more, fifty or more, one hundred or more, or one thousand or more dose(s). In some embodiments, the dose comprises at least 25 mg, at least 50 mg, at least 75 mg, at least 100 mg, at least 125 mg, at least 150 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 550 mg, at least 600 mg, at least 650 mg, at least 700 mg, at least 750 mg, at least 800 mg, or at least 850 mg of a compound of Formula I or Formula II (e.g., nicotinamide riboside). In some embodiments, the dose comprises at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 90 mg, at least 95 mg, at least 100 mg, at least 110 mg, at least 120 mg, at least 130 mg, at least 140 mg, at least 150 mg, at least 160 mg, least 170 mg, at least 180 mg, at least 190 mg, at least 200 mg, or at least 250 mg of a compound of formula III (e.g., pterostilbene).

The compositions disclosed herein may be administered over any period of time effective to achieve the desired therapeutic response for a particular patient, composition, and mode of administration, without being toxic to the patient. The period of time may be at least 1 day, at least 10 days, at least 20 days, at least 30, days, at least 60 days, at least three months, at least six months, at least a year, at least three years, at least five years, or at least ten years. The dose may be administered when needed, sporadically, or at regular intervals. For example, the dose may be administered monthly, weekly, biweekly, triweekly, once a day, or twice a day.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating cystic fibrosis in a subject comprising administering to the subject a composition comprising nicotinamide riboside.

2. The method of claim 1, wherein the composition further comprises pterostilbene.

3. A method of treating and/or preventing a symptom of cystic fibrosis in a subject comprising administering to the subject a composition comprising nicotinamide riboside.

4. The method of claim 1, wherein the composition further comprises pterostilbene.

5. The method of claim 3 or 4, wherein the symptom is a respiratory symptom of cystic fibrosis.

6. The method of claim 5, wherein the respiratory symptom is sticky mucus buildup in lungs, inflammation in the lungs, atelectasis, lung infections, chronic cough, wheezing, collapsed lung, nasal polyps and/or sinusitis.

7. The method of claim 3 or 4, wherein the symptom is a digestive symptom of cystic fibrosis.

8. The method of claim 7, wherein the digestive symptom is bowel obstruction, inflammation of the GI tract, fatty stools, severe constipation, rectal prolapse, pancreatitis, or biliary cirrhosis.

9. The method of claim 4 or 5, wherein the symptom is a reproductive tract symptom.

10. The method of claim 9, wherein the reproductive tract symptom is blockage of the vas deferens.

11. A method of increasing ion channel transport in epithelial cells in the lungs, digestive tract, or reproductive tract of a subject with cystic fibrosis, comprising administering to the subject a composition comprising nicotinamide riboside.

12. The method of claim 11, wherein the composition further comprises pterostilbene.

13. A method of reducing sticky mucus in the lungs, digestive tract, or reproductive tract in a subject with cystic fibrosis, comprising administering to the subject a composition comprising nicotinamide riboside.

14. The method of claim 13, wherein the composition further comprises pterostilbene.

15. A method of treating a CFTR-related disease or disorder in a subject comprising administering to the subject a composition comprising nicotinamide riboside.

16. The method of claim 15, wherein the composition further comprises pterostilbene.

17. The method of claim 15 or 16, wherein the disease or disorder is acute, recurrent, or chronic pancreatitis or acute, recurrent, or chronic bronchiectasis.

18. The method of any one of the preceding claims, wherein the subject carries a CFTR gating mutant allele or a CFTR conductance mutant allele.

19. The method of any one of the preceding claims, wherein the method comprises conjointly administering a second therapeutic for cystic fibrosis.

20. The method of claim 19, wherein the second therapeutic is a CFTR potentiator, a mucoactive agent, an antibiotic, an inhaled beta-agonist, or an anti-inflammatory agent.

21. The method of any one of claims 1 to 20, wherein the administration of the composition comprises administering one or more doses of the composition.

22. The method of claim 21, wherein each dose of the composition comprises at least 200 mg of nicotinamide riboside.

23. The method of claim 21, wherein each dose of the composition comprises at least 250 mg of nicotinamide riboside.

24. The method of claim 21, wherein each dose of the composition comprises at least 300 mg of nicotinamide riboside.

25. The method of claim 21, wherein each dose of the composition comprises at least 350 mg of nicotinamide riboside.

26. The method of claim 21, wherein each dose of the composition comprises at least 400 mg of nicotinamide riboside.

27. The method of claim 21, wherein each dose of the composition comprises at least 450 mg of nicotinamide riboside.

28. The method of claim 21, wherein each dose of the composition comprises at least 500 mg of nicotinamide riboside.

29. The method of claim 21, wherein each dose of the composition comprises at least 550 mg of nicotinamide riboside.

30. The method of any one of claims 21 to 29, wherein each dose of the composition comprises at least 15 mg of pterostilbene.

31. The method of any one of claims 21 to 29, wherein each dose of the composition comprises at least 25 mg of pterostilbene.

32. The method of any one of claims 21 to 29, wherein each dose of the composition comprises at least 50 mg of pterostilbene.

33. The method of any one of claims 21 to 29, wherein each dose of the composition comprises at least 75 mg of pterostilbene.

34. The method of any one of claims 21 to 29, wherein each dose of the composition comprises at least 100 mg of pterostilbene.

35. The method of any one of claims 21 to 29, wherein each dose of the composition comprises at least 125 mg of pterostilbene.

36. The method of any one of claims 21 to 29, wherein each dose of the composition comprises at least 150 mg of pterostilbene.

37. The method of any one of claims 21 to 36, wherein two or more doses of the composition are administered.

38. The method of any one of claims 21 to 37, wherein thirty or more doses of the composition are administered.

39. The method of any one of claims 21 to 38, wherein fifty or more doses of the composition are administered.

40. The method of any one of claims 21 to 39, wherein one hundred or more doses of the composition are administered.

41. The method of any one of claims 21 to 40, wherein the dose of the composition is administered at least once a week.

42. The method of any one of claims 21 to 40, wherein the dose is administered at least twice a week.

43. The method of any one of claims 21 to 40, wherein the dose is administered at least three times a week.

44. The method of any one of claims 21 to 40, wherein the dose is administered at least once a day.

45. The method of any one of claims 21 to 40, wherein the dose is administered at least twice a day.

46. The method of any one of claims 41 to 45, wherein the doses are administered for at least 7 days.

47. The method of any one of claims 41 to 45, wherein the doses are administered for at least 30 days.

48. The method of any one of claims 41 to 45, wherein the doses are administered for at least 60 days.

49. The method of any one of claims 41 to 45, wherein the doses are administered for at least 90 days.

50. The method of any one of claims 1 to 49, wherein the composition is formulated as a pill, a tablet, or a capsule.

51. The method of any one of claims 1 to 50, wherein the composition is administered orally.

52. The method of any one of claims 1 to 51, wherein the composition is self-administered.
